(19) United States
(12) Patent Application Publication
(10) Pub. No.: US 2026/0102169 A1
(43) Pub. Date: Apr. 16, 2026

(54) ORTHOPAEDIC SURGICAL INSTRUMENT AND METHOD OF LOCATING A CUTTING BLOCK ON A PATIENT'S BONE

(71) Applicant: DePuy Ireland Unlimited Company, Ringaskiddy (IE)

(72) Inventors: Kyle C. Helm, Fort Wayne, IN (US); Anees Ahamed Muhamed Farook, Warsaw, IN (US)

(21) Appl. No.: 19/345,226

(22) Filed: Sep. 30, 2025

Related U.S. Application Data
(60) Provisional application No. 63/707,280, filed on Oct. 15, 2024.

(51) Int. Cl. A61B 17/15; A61B 17/00

(52) U.S. Cl. CPC A61B 17/157; A61B 2017/00477

(57) ABSTRACT

An orthopaedic surgical instrument and method are disclosed. The orthopaedic surgical instrument is configured to locate a tibial cutting block on a patient’s bone to guide resection of the patient’s bone and prepare it to receive an orthopaedic prosthetic component.

This application claims priority to U.S. Provisional Patent Application Serial No. 63/707,280, which was filed on October 15, 2024, which is hereby incorporated by reference in its entirety.

### TECHNICAL FIELD

The present disclosure relates generally to surgical instruments and methods and, more particularly, to orthopaedic surgical instruments and methods for use in a performance of a knee replacement procedure.

### BACKGROUND

Joint arthroplasty is a well-known surgical procedure by which a diseased and/or damaged natural joint is replaced by a prosthetic joint. For example, in a knee arthroplasty surgical procedure, a patient’s natural knee joint is partially or totally replaced by a prosthetic knee joint or knee prosthesis. A typical knee prosthesis includes a tibial tray, a femoral component, and a polymer insert or bearing positioned between the tibial tray and the femoral component. The tibial tray generally includes a plate having a stem extending distally therefrom, and the plate is configured to engage a planar surface of the patient’s tibia, which is created by resecting a portion of the proximal end of the patient’s tibia.

A proximal tibial cutting block is one tool a surgeon may use to guide the resection of the patient’s tibia. Such cutting blocks may include one or more cutting guides that define resection planes. Various tools are available to locate the cutting block relative to the patient’s tibia and thereby position the cutting guide of the cutting block in a desired location. One such tool is an Adjustable Tibial Stylus, which is commercially available from DePuy Synthes Products, Inc. of Warsaw, Indiana. Such a stylus may be used to locate the cutting guide of the cutting block at a desired distance from a reference point of the proximal end of the patient’s tibia, thereby positioning the resection plane of the cutting guide along the longitudinal axis of the patient’s tibia such that the desired amount of bone may be removed to create the planar surface.

As shown in FIG. 1A, the resection plane 10 may be angled in an anterior-posterior direction to create a planar surface on the proximal end 16 of the patient’s tibia 18 having a posterior slope. Additionally, as shown in FIG. 1B, the resection plane 10 may be angled in a medial-lateral direction, with a high lateral side 12 and a low medial side 14, to create a planar surface that is angled and has a medial-lateral slope when viewed from the front of the patient.

### SUMMARY

An orthopaedic surgical instrument and method are shown and described. The orthopaedic surgical instrument includes a frame including a plate configured to be received in a cutting guide of a tibial cutting block and an arm coupled to the frame. The arm extends along a longitudinal axis from a first end to a second end including a distal tip configured to contact a proximal surface of a patient’s tibia. The orthopaedic surgical instrument also includes a pin having a pin end movably mounted to the frame and engaged with the arm. The arm includes a curved surface and a groove that extends along the longitudinal axis, and the arm is rotatable about the longitudinal axis between a first position in which the pin end is received in the groove to resist rotation of the arm, and a second position in which the pin end engages the curved surface such that the arm is permitted to rotate freely.

In some embodiments, the groove may be connected to the curved surface. The frame may include a channel sized to receive the arm. Additionally, in some embodiments, the channel may include a curved surface that matches the curved surface of the arm.

In some embodiments, the frame may further include an elongated pin, and the channel and the elongated pin may cooperate to couple the arm to the frame.

In some embodiments, the orthopaedic surgical instrument may further include a height adjustment mechanism to change a distance between the plate and the arm. In some embodiments, the frame may include a housing configured to rotate relative to the plate about an axis extending perpendicular to the longitudinal axis.

According to another aspect, a method of surgically preparing a patient’s tibia during an orthopaedic surgical knee procedure includes assembling a tibial cutting block to a base of an orthopaedic surgical instrument, the tibial cutting block having a mediolaterally extending cutting slot. The method also includes positioning the tibial cutting block against the patient’s tibia. The method also includes operating the orthopaedic surgical instrument to position a distal tip of an arm of the orthopaedic surgical instrument in contact with a distal-most point of one of a medial compartment and a lateral compartment of the patient’s tibia. The method also includes rotating the arm of the orthopaedic surgical instrument relative to the tibial cutting block such that the distal tip moves from an inferiorly-extending position to another position. The method also includes moving the arm in a mediolateral direction relative to the tibial cutting block while the base is assembled with the tibial cutting block and the tibial cutting block positioned against the patient’s tibia. The method also includes rotating the arm of the orthopaedic surgical instrument relative to the tibial cutting block such that the distal tip moves to the inferiorly-extending position. The method also includes operating the orthopaedic surgical instrument to position the distal tip of the arm of the orthopaedic surgical instrument in contact with the distal-most point of the other one of the medial compartment and the lateral compartment of the patient’s tibia.

In some embodiments, positioning the tibial cutting block against the patient’s tibia may further include positioning the assembled tibial cutting block and the orthopaedic surgical instrument on the patient’s tibia such that a bone facing surface of the tibial cutting block abuts an anterior surface of the patient’s tibia. The method may include, before rotating the arm of the orthopaedic surgical instrument relative to the tibial cutting block such that the distal tip moves from the inferiorly-extending position, inserting a first fixation pin into the tibial cutting block to partially attach the tibial cutting block to the patient’s tibia.

In some embodiments, operating the orthopaedic surgical instrument to position the distal tip of the arm of the orthopaedic surgical instrument in contact with the distal-most point of the other one of the medial compartment and the lateral compartment of the patient’s tibia may include pivoting the tibial cutting block about the first fixation pin to adjust a medial-lateral slope of the cutting slot.

In some embodiments, the method may include, after operating the orthopaedic surgical instrument to position the distal tip of the arm of the orthopaedic surgical instrument in contact with the distal-most point of the other one of the medial compartment and the lateral compartment of the patient’s tibia, inserting a second fixation pin into the tibial cutting block to attach the tibial cutting block to the patient’s tibia.

In some embodiments, rotating the arm of the orthopaedic surgical instrument relative to the tibial cutting block such that the distal tip moves from the inferiorly-extending position may include moving an end of a retaining pin of the orthopaedic surgical instrument out of a groove formed in the arm. Rotating the arm of the orthopaedic surgical instrument relative to the tibial cutting block such that the distal tip moves to the inferiorly-extending position may include moving the end of the retaining pin of the orthopaedic surgical instrument into the groove formed in the arm.

In some embodiments, rotating the arm of the orthopaedic surgical instrument relative to the tibial cutting block such that the distal tip moves to the inferiorly-extending position may include moving the end of the retaining pin out of contact with a convex outer surface of the arm into the groove formed in the arm. The method may include, operating, with the distal tip of the arm in contact with the distal-most point of the one of the medial compartment and the lateral compartment of the patient’s tibia, a height adjustment mechanism to adjust a position of the cutting slot relative to the patient’s tibia.

In some embodiments, the method may include operating, with the distal tip of the arm in contact with the distal-most point of the other one of the medial compartment and the lateral compartment of the patient’s tibia, a height adjustment mechanism to alter an angle of the cutting slot relative to an anterior surface of the patient’s tibia. Operating the orthopaedic surgical instrument to position the distal tip of the arm of the orthopaedic surgical instrument in contact with the distal-most point of the one of the medial compartment and the lateral compartment of the patient’s tibia may include rotating a cap of a height adjustment mechanism to position the distal tip of the arm of the orthopaedic surgical instrument in contact with the distal-most point of the one of the medial compartment and the lateral compartment of the patient’s tibia.

In some embodiments, operating the orthopaedic surgical instrument to position the distal tip of the arm of the orthopaedic surgical instrument in contact with the distal-most point of the other one of the medial compartment and the lateral compartment of the patient’s tibia may include rotating the cap to position the distal tip of the arm of the orthopaedic surgical instrument in contact with the distal-most point of the other one of the medial compartment and the lateral compartment of the patient’s tibia.

In some embodiments, the method may include sliding the arm to move the distal tip in an anterior-posterior direction relative to the base of the orthopaedic surgical instrument to change the position of the distal tip relative to the one of the medial compartment and the lateral compartment of the patient’s tibia. Rotating the arm of the orthopaedic surgical instrument relative to the tibial cutting block such that the distal tip moves from the inferiorly-extending position may include rotating a handle coupled to the arm opposite the distal tip thereof.

### BRIEF DESCRIPTION OF THE DRAWINGS

The detailed description particularly refers to the following figures, in which:

FIG. 1A is a sagittal elevation view of a tibia;

FIG. 1B is an anterior elevation view of the tibia of FIG. 1A;

FIG. 2 is a perspective view of an orthopaedic surgical instrument;

FIG. 3 is a perspective view of a portion of the orthopaedic surgical instrument of FIG. 2;

FIG. 4 is a perspective view of another portion of the orthopaedic surgical instrument of FIG. 2;

FIG. 5 is a perspective view of the orthopaedic surgical instrument of FIG. 2 mounted to a tibial cutting block with a stylus arm of the orthopaedic surgical instrument located in a first rotational position; and

FIG. 6 is a perspective view of the orthopaedic surgical instrument of FIG. 2 mounted to the tibial cutting block with the stylus arm located in a second rotational position.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, proximal, distal, etcetera, may be used throughout the specification in reference to the orthopaedic implants and surgical instruments described herein as well as in reference to the patient’s natural anatomy. Such terms have well-understood meanings in both the study of anatomy and the field of orthopaedics. Use of such anatomical reference terms in the written description and claims is intended to be consistent with their well-understood meanings unless noted otherwise.

The exemplary embodiments of the present disclosure are described and illustrated below to encompass surgical instruments and methods for knee replacement surgeries. It will also be apparent to those of ordinary skill in the art that the preferred embodiments discussed below are exemplary in nature and may be reconfigured without departing from the scope and spirit of the present invention. However, for clarity and precision, the exemplary embodiments as discussed below may include optional steps, methods, and features that one of ordinary skill should recognize as not being a requisite to fall within the scope of the present invention.

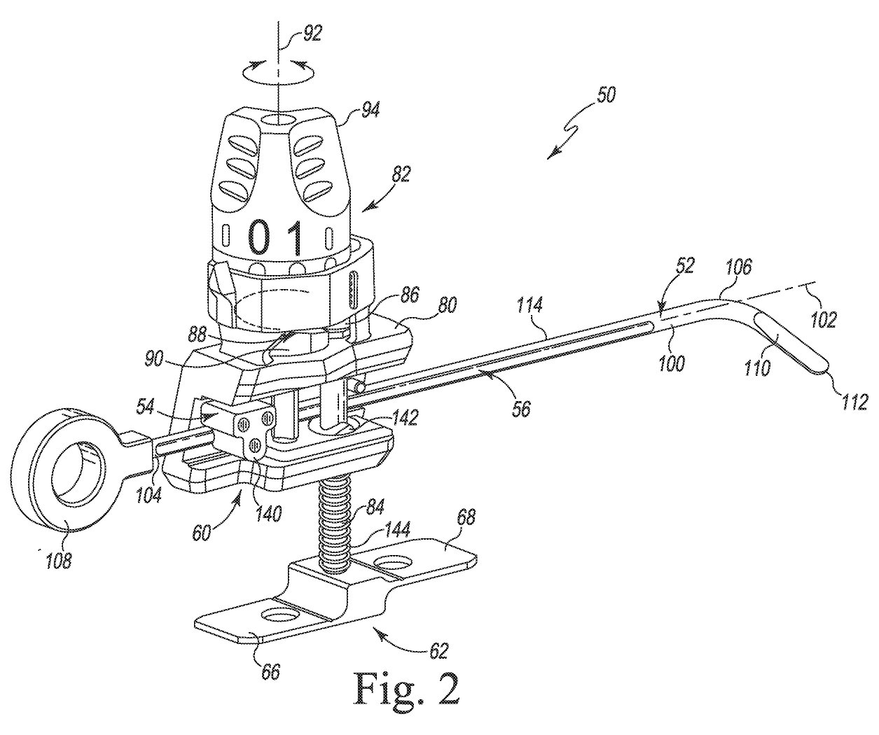

Referring now to FIG. 2, an orthopaedic surgical instrument 50 is shown. The orthopaedic surgical instrument 50 includes a rotatable stylus arm 52 that is configured to be rotated into and out of engagement with the proximal end 16 of the patient’s tibia 18 and a locking mechanism 54 configured to engage the stylus arm 52, particularly a groove 56 defined in the stylus arm 52, to resist rotation of the stylus arm 52, as described in greater detail below.

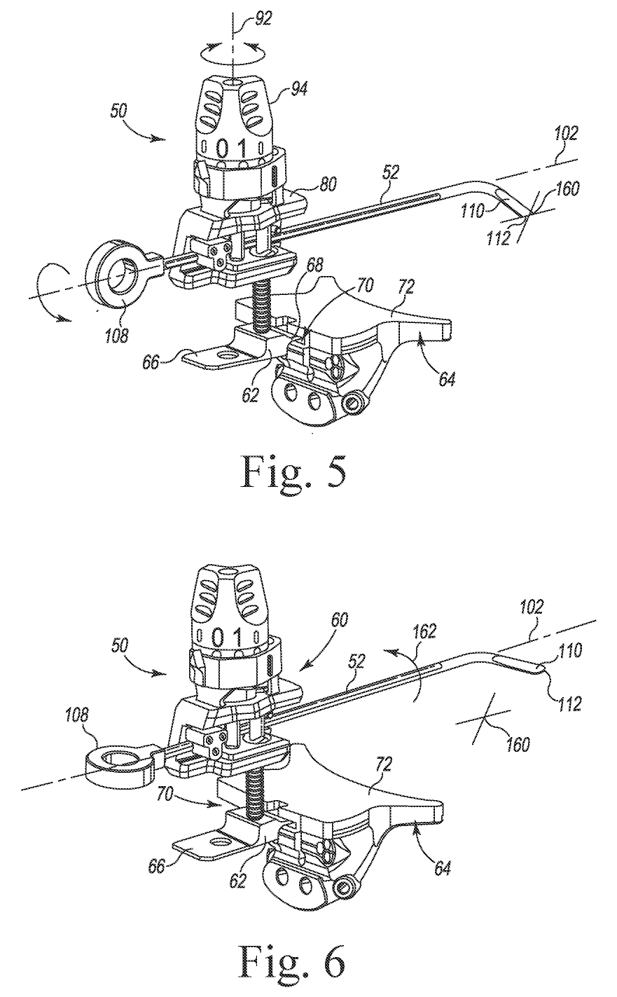

The rotatable stylus arm 52 is coupled to a frame 60 of the orthopaedic surgical instrument 50, as shown in FIG. 2. The frame 60 includes a foot 62, which is positioned below the stylus arm 52 and is configured to be coupled to a tibial cutting block 64 (see FIG. 5) to attach the orthopaedic surgical instrument 50 to the tibial cutting block 64. In the illustrative embodiment, the foot 62 includes a plate 66 and a plate 68 extending in opposite directions. Each of the plates 66, 68 is configured to be inserted into a slot 70 (see FIG. 5) of the tibial cutting block 64. As shown in FIG. 2, the plate 68 is positioned above the plate 66 by a predetermined distance, permitting the surgeon to position the stylus arm 52 at different distances from a cutting guide (shown in FIG. 5 as a cutting surface 72) of the tibial cutting block 64.

As shown in FIG. 2, the frame 60 also includes a housing 80 that supports the stylus arm 52 and a height adjustment mechanism 82 configured to adjust the position of the foot 62 relative to the housing 80 (and hence the stylus arm 52). The height adjustment mechanism 82 includes a shaft 84 attached to the foot 62 and extending into the housing 80. The shaft 84 is rotatably coupled to the housing 80 such that the housing 80 may rotate about the shaft 84 relative to the foot 62.

The shaft 84 includes a tooth or flange 86 positioned in the housing 80, which is received in a helical groove 88 defined in a dial 90. The helical groove 88, which is partially shown in broken line in FIG. 2, extends around the dial 90 such that two groove ends of the helical groove 88 are offset vertically from each other. The dial 90 is coupled to the housing 80 and rotatable about an axis 92 extending upwardly through a cap 94. The cap 94 is attached to the dial 90 and is sized and shaped to be grasped by the surgeon and rotated about the axis 92. When the surgeon rotates the cap 94, the dial 90 rotates with the cap 94, advancing the flange 86 along the helical groove 88 between the two groove ends. Depending on the direction of rotation, the flange 86 is moved up or down the helical groove 88, thereby changing the distance between the foot 62 and the housing 80 and moving them closer to or farther away from each other.

Returning to the stylus arm 52, the stylus arm 52 includes an elongated shaft 100 that extends along a longitudinal axis 102 from a proximal end 104 to a distal end 106, as shown in FIG. 2. A knob or handle 108 is attached at the proximal end 104 of the elongated shaft 100, while a stylus tip 110 extends at an obtuse angle from the distal end 106 of the elongated shaft 100 to a lower edge 112. The stylus tip 110 is configured to be brought into and out of engagement with the proximal end 16 of the patient’s tibia 18 to assist in locating the tibial cutting block 64, as described in greater detail below.

The elongated shaft 100 includes a curved outer surface 114 that extends along the longitudinal axis 102, as shown in FIGS. 2 and 4. The groove 56, also extending along the longitudinal axis 102, is defined in the elongated shaft 100. In the illustrative embodiment, the groove 56 is sized to receive an end 120 (see FIG. 4) of a retaining pin 122. The engagement between the end 120 of the retaining pin 122 and a surface 124 defining the groove 56 resists the rotation of the stylus arm 52 about the longitudinal axis 102. It should be appreciated that in other embodiments the groove 56 may be omitted, and the end 120 of the retaining pin 122 may engage the curved outer surface 114. In such embodiments, a spring of the retaining pin 122 may be sized to impart sufficient force to resist rotation of the shaft 100. It should be further appreciated that in other embodiments one or more spring-loaded members such as, for example, a cantilevered arm attached to frame 60, may engage the elongated shaft 100 to resist rotation, thereby replacing the retaining pin 122 and the bracket 140 in the locking mechanism 54.

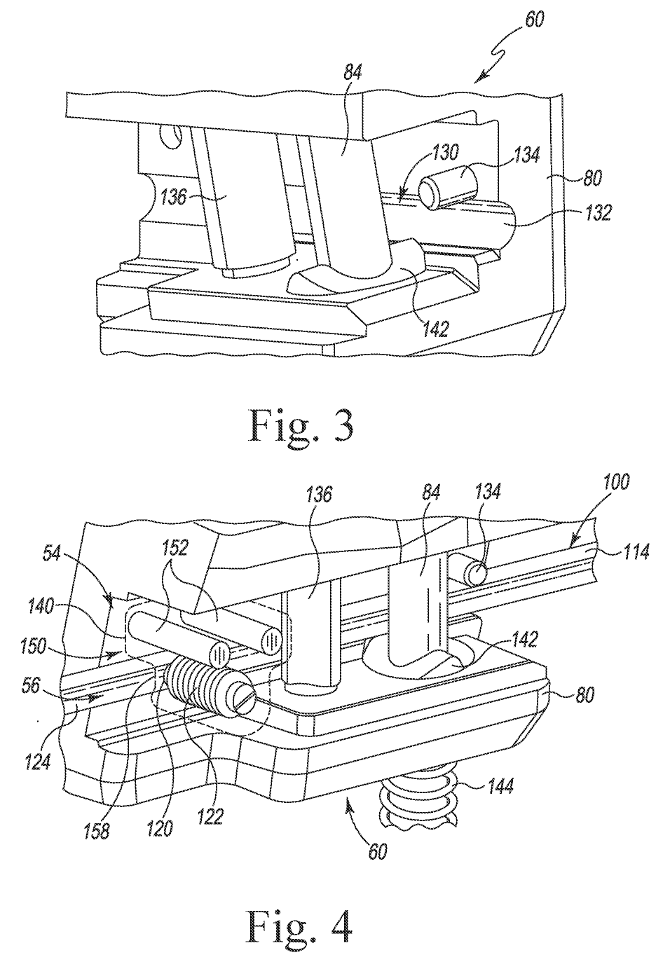

Referring now to FIG. 3, the housing 80 includes a channel 130 that is sized to receive the elongated shaft 100 therein. In the illustrative embodiment, a curved surface 132, which is sized and shaped to match the curved outer surface 114 of the elongated shaft 100, defines the channel 130. The frame 60 also includes an elongated pin 134 that is positioned above the channel 130 and a connecting shaft 136 that is positioned opposite the channel 130, adjacent to the shaft 84 of the height adjustment mechanism 82. The shaft 84, the connecting shaft 136, the channel 130, and the elongated pin 134 cooperate with a bracket 140 (described below) to retain the stylus arm 52 in the frame 60.

The frame 60 further includes a floating collar 142 that is positioned on the shaft 84 in an opening defined in the housing 80, as shown in FIGS. 3 and 4. As shown in FIGS. 2 and 4, a spring 144 positioned between the floating collar 142 and the foot 62 biases the floating collar 142 into contact with the elongated shaft 100 of the stylus arm 52 thereby causing engagement of the elongated shaft 100 with the elongated pin 134.

Referring now to FIG. 4, the bracket 140 includes a lower channel 150 that receives the elongated shaft 100 of the stylus arm 52. A pair of fasteners 152, illustratively elongated pins, secure the bracket 140 to the housing 80. The retaining pin 122 is attached to the bracket 140 below the fasteners 152. In the illustrative embodiment, the retaining pin 122 includes a spring (not shown) that biases the end 120 of the retaining pin 122 away from an inner surface 158 of the bracket 140 and into engagement with the elongated shaft 100 of the stylus arm 52. In the illustrative embodiment, the bracket 140 and the retaining pin 122 form the locking mechanism 54 configured to engage the stylus arm 52 to resist and/or block rotation of the stylus arm 52, as described in greater detail below.

Referring now to FIGS. 5-6, the surgeon may mount the orthopaedic surgical instrument 50 to the tibial cutting block 64 by inserting the plate 68 of the foot 62 into the slot 70 of the tibial cutting block 64. In illustrative embodiments, the slot 70 is a mediolaterally extending cutting slot. With the stylus tip 110 pointed toward the patient’s tibia 18, as shown in FIG. 5, the surgeon may operate the height adjustment mechanism 82, rotating the cap 94 as described above to change the distance between the housing 80 and the foot 62 and thereby moving the lower edge 112 of the stylus tip 110 into contact with a point 160 on the proximal end 16 of the patient’s tibia 18. For the lower edge 112 to make contact with the point 160, the stylus arm 52 is in a first position, as shown in FIG. 5, such that the stylus tip 110 is in an inferiorly-extending position. In this way, the stylus tip 110 faces toward the proximal end 16 of the patient’s tibia 18. The point 160 may, for example, be located at a distal-most point of a medial compartment of the patient’s tibia 18.

While holding the tibial cutting block 64 in position on the patient’s tibia 18, the surgeon may grasp the handle 108 to rotate the stylus arm 52 about the longitudinal axis 102, as suggested in FIGS. 5 and 6. As the surgeon rotates the handle 108, the end 120 of the retaining pin 122 moves out of the groove 56 of the elongated shaft 100 of the stylus arm 52, and the engagement between the curved outer surface 114 of the stylus arm 52 and the end 120 of the retaining pin 122 depresses the spring of the retaining pin 122, thereby allowing the end 120 of the retaining pin 122 to move toward the inner surface 158 of the bracket 140. The engagement between the end 120 of the retaining pin 122 and the curved outer surface 114 of the stylus arm 52 allows the surgeon to freely rotate the stylus arm 52 about the longitudinal axis 102, thereby disengaging the lower edge 112 of the stylus tip 110 from the point 160 on the proximal end 16 of the patient’s tibia 18, as shown in FIG. 6. Rotation of the stylus arm 52 about the longitudinal axis 102 moves the stylus arm 52 from the first position, as shown in FIG. 5, to a second position, as shown in FIG. 6. In the second position of the stylus arm 52, the stylus tip 110 is in a mediolaterally-extending position, a superiorly-extending position, or a non-inferiorly-extending position.

With the lower edge 112 of the stylus tip 110 out of contact with the proximal end 16 of the patient’s tibia 18, the surgeon may rotate the housing 80 (and the stylus arm 52) about the shaft 84, as indicated by arrow 162 in FIG. 6, relative to the foot 62, which remains stationary with the tibial cutting block 64 on the patient’s tibia 18. This mediolateral movement allows the surgeon to reposition the stylus arm 52 over, for example, the lateral compartment of the patient’s tibia 18. During movement of the stylus arm 52 along arrow 162, the stylus tip 110 faces away from the proximal end 16 of the patient’s tibia 18. With the lower edge 112 of the stylus tip 110 out of contact with the proximal end 16 of the patient’s tibia 18, the surgeon may also slide the stylus arm 52 relative to the housing 80 to move the stylus tip 110 in an anterior-posterior direction relative to the foot 62. This anterior-posterior movement allows the surgeon to reposition the stylus arm 52 over, for example, the lateral compartment of the patient’s tibia 18.

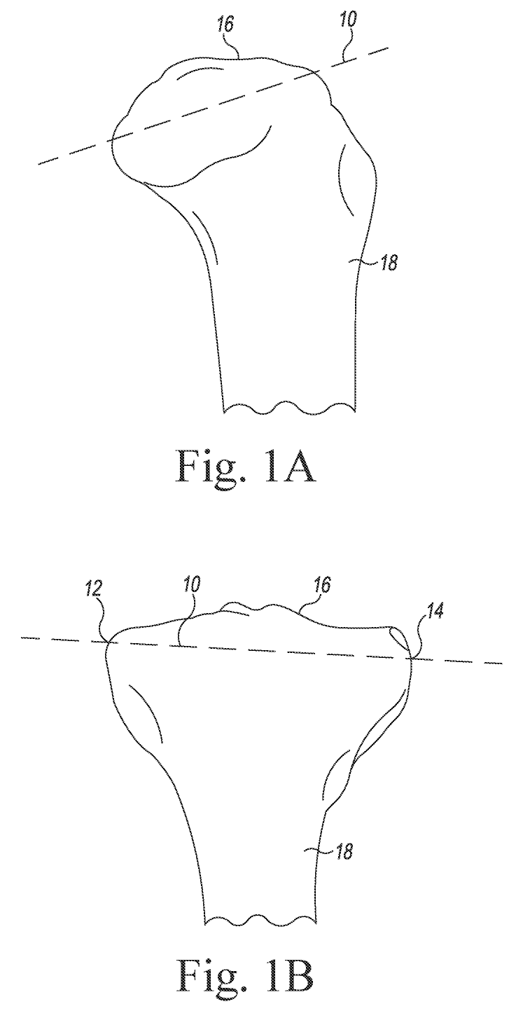

The surgeon may use the handle 108 to rotate the stylus arm 52 about the longitudinal axis 102. As the surgeon rotates the handle 108, the end 120 of the retaining pin 122 is advanced back toward the groove 56 of the elongated shaft 100, with the spring of the retaining pin 122 urging the end 120 of the retaining pin 122 into the groove 56 such that further rotation of the stylus arm 52 is resisted and/or blocked. With the lower edge 112 of the stylus tip 110 in contact with the lateral compartment of the patient’s tibia 18, the surgeon may evaluate the resulting position of the cutting surface 72 of the tibial cutting block 64, which may angle the resection plane 10, as shown in FIG. 1B. If the surgeon is satisfied with the position, the tibial cutting block 64 may be attached to the patient’s tibia 18 via fixation pins before the orthopaedic surgical instrument 50 is removed and the resection performed.

Prior to rotating the stylus arm 52 and moving the lower edge 112 of the stylus tip 110 from one compartment of the patient’s tibia 18 to the other, the surgeon may insert a fixation pin in one guide hole of the tibial cutting block 64 to partially attach the tibial cutting block 64 to the patient’s tibia 18. As the surgeon moves the lower edge 112 of the stylus tip 110 to the other compartment, the tibial cutting block 64 is permitted to pivot about the fixation pin, assisting the surgeon in setting the medial-lateral slope of the resection plane 10.

It should be appreciated that the orthopaedic surgical instrument 50 may be attached to cutting blocks having configurations different from that of the tibial cutting block 64. For example, the plates 66, 68 of the foot 62 may be sized and shaped to be received in captured or closed cutting guide slots of a cutting block rather than in a separate slot 70.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure.

1. An orthopaedic surgical instrument, comprising:
a frame including a plate configured to be received in a cutting guide of a tibial cutting block,
an arm coupled to the frame, the arm extends along a longitudinal axis from a first end to a second end, the second end including a distal tip configured to contact a proximal surface of a patient’s tibia, and
a pin having a pin end movably mounted to the frame and engaged with the arm,
wherein the arm includes a curved surface and a groove extending along the longitudinal axis, and
wherein the arm is rotatable about the longitudinal axis between (i) a first position in which the pin end is received in the groove to resist rotation of the arm, and (ii) a second position in which the pin end engages the curved surface such that the arm is permitted to rotate freely.

2. The orthopaedic surgical instrument of claim 1, wherein the groove is connected to the curved surface.

3. The orthopaedic surgical instrument of claim 1, wherein the frame includes a channel sized to receive the arm.

4. The orthopaedic surgical instrument of claim 3, wherein the channel includes a curved surface that matches the curved surface of the arm.

5. The orthopaedic surgical instrument of claim 3, wherein the frame further includes an elongated pin, and the channel and the elongated pin cooperate to couple the arm to the frame.

6. The orthopaedic surgical instrument of claim 1, further comprising a height adjustment mechanism to change a distance between the plate and the arm.

7. The orthopaedic surgical instrument of claim 6, wherein the frame includes a housing configured to rotate relative to the plate about an axis extending perpendicular to the longitudinal axis.

8. A method of surgically preparing a patient’s tibia during an orthopaedic surgical knee procedure, comprising:
assembling a tibial cutting block to a base of an orthopaedic surgical instrument, the tibial cutting block having a mediolaterally extending cutting slot,
positioning the tibial cutting block against the patient’s tibia,
operating the orthopaedic surgical instrument to position a distal tip of an arm of the orthopaedic surgical instrument in contact with a distal-most point of one of a medial compartment and a lateral compartment of the patient’s tibia,
rotating the arm of the orthopaedic surgical instrument relative to the tibial cutting block such that the distal tip moves from an inferiorly-extending position to another position,
moving the arm in a mediolateral direction relative to the tibial cutting block while the base is assembled with the tibial cutting block and the tibial cutting block positioned against the patient’s tibia,
rotating the arm of the orthopaedic surgical instrument relative to the tibial cutting block such that the distal tip moves to the inferiorly-extending position, and
operating the orthopaedic surgical instrument to position the distal tip of the arm of the orthopaedic surgical instrument in contact with the distal-most point of the other one of the medial compartment and the lateral compartment of the patient’s tibia.

9. The method of claim 8, wherein positioning the tibial cutting block against the patient’s tibia further comprises positioning the assembled tibial cutting block and the orthopaedic surgical instrument on the patient’s tibia such that a bone facing surface of the tibial cutting block abuts an anterior surface of the patient’s tibia.

10. The method of claim 8, further comprising, before rotating the arm of the orthopaedic surgical instrument relative to the tibial cutting block such that the distal tip moves from the inferiorly-extending position, inserting a first fixation pin into the tibial cutting block to partially attach the tibial cutting block to the patient’s tibia.

11. The method of claim 10, wherein operating the orthopaedic surgical instrument to position the distal tip of the arm of the orthopaedic surgical instrument in contact with the distal-most point of the other one of the medial compartment and the lateral compartment of the patient’s tibia comprises pivoting the tibial cutting block about the first fixation pin to adjust a medial-lateral slope of the cutting slot.

12. The method of claim 10, further comprising, after operating the orthopaedic surgical instrument to position the distal tip of the arm of the orthopaedic surgical instrument in contact with the distal-most point of the other one of the medial compartment and the lateral compartment of the patient’s tibia, inserting a second fixation pin into the tibial cutting block to attach the tibial cutting block to the patient’s tibia.

13. The method of claim 8, wherein rotating the arm of the orthopaedic surgical instrument relative to the tibial cutting block such that the distal tip moves from the inferiorly-extending position comprises moving an end of a retaining pin of the orthopaedic surgical instrument out of a groove formed in the arm.

14. The method of claim 13, wherein rotating the arm of the orthopaedic surgical instrument relative to the tibial cutting block such that the distal tip moves to the inferiorly-extending position comprises moving the end of the retaining pin of the orthopaedic surgical instrument into the groove formed in the arm.

15. The method of claim 14, wherein rotating the arm of the orthopaedic surgical instrument relative to the tibial cutting block such that the distal tip moves to the inferiorly-extending position comprises moving the end of the retaining pin out of contact with a convex outer surface of the arm into the groove formed in the arm.

16. The method of claim 8, further comprising, operating, with the distal tip of the arm in contact with the distal-most point of the one of the medial compartment and the lateral compartment of the patient’s tibia, a height adjustment mechanism to adjust a position of the cutting slot relative to the patient’s tibia.

17. The method of claim 8, further comprising, operating, with the distal tip of the arm in contact with the distal-most point of the other one of the medial compartment and the lateral compartment of the patient’s tibia, a height adjustment mechanism to alter an angle of the cutting slot relative to an anterior surface of the patient’s tibia.

18. The method of claim 8, wherein:
operating the orthopaedic surgical instrument to position the distal tip of the arm of the orthopaedic surgical instrument in contact with the distal-most point of the one of the medial compartment and the lateral compartment of the patient’s tibia comprises rotating a cap of a height adjustment mechanism to position the distal tip of the arm of the orthopaedic surgical instrument in contact with the distal-most point of the one of the medial compartment and the lateral compartment of the patient’s tibia, and
operating the orthopaedic surgical instrument to position the distal tip of the arm of the orthopaedic surgical instrument in contact with the distal-most point of the other one of the medial compartment and the lateral compartment of the patient’s tibia comprises rotating the cap to position the distal tip of the arm of the orthopaedic surgical instrument in contact with the distal-most point of the other one of the medial compartment and the lateral compartment of the patient’s tibia.

19. The method of claim 8, further comprising sliding the arm to move the distal tip in an anterior-posterior direction relative to the base of the orthopaedic surgical instrument to change the position of the distal tip relative to the one of the medial compartment and the lateral compartment of the patient’s tibia.

20. The method of claim 8, wherein rotating the arm of the orthopaedic surgical instrument relative to the tibial cutting block such that the distal tip moves from the inferiorly-extending position comprises rotating a handle coupled to the arm opposite the distal tip thereof.
